(19) United States
(12) Patent Application Publication
(10) Pub. No.: US 2024/0041890 A1
(43) Pub. Date: Feb. 8, 2024

(54) ANTI-HAIR-LOSS COMPOSITION

(71) Applicant: WELLA GERMANY GMBH, Darmstadt (DE)

(72) Inventors: Christian Springob, Lorsch (DE); Massimo Morini, Bad Soden (DE)

(73) Assignee: WELLA GERMANY GMBH, Darmstadt (DE)

(21) Appl. No.: 18/255,027

(22) PCT Filed: Nov. 25, 2021
(86) PCT No.: PCT/EP2021/083009

§ 371 Date: May 30, 2023

Related U.S. Application Data
(60) Provisional application No. 63/119,432, filed on Nov. 30, 2020.

(51) Int. Cl. A61K 31/522; A61K 31/045; A61K 47/12; A61K 45/06; A61K 47/10; A61K 31/455; A61P 17/14

(52) U.S. Cl. CPC A61K 31/522; A61K 31/045; A61K 47/12; A61K 45/06; A61K 47/10; A61K 31/455; A61P 17/14

(57) ABSTRACT

A composition for promoting hair growth, and/or reducing or preventing hair loss, respectively, comprises sandal pentanol, caffeine, one or more saturated and unsaturated C10-C18 fatty acids, and a solvent.

### FIELD OF THE INVENTION

The present invention relates to a cosmetic composition, especially a hair tonic, for reducing or treating hair loss. The composition contains an active ingredient combination of sandal pentanol, caffeine, and one or more saturated or unsaturated C10-C18-fatty acids, or salts thereof. The subject matter of the present invention also includes a cosmetic method for promoting natural hair growth and reducing hair loss.

### BACKGROUND OF THE INVENTION

The growth of human hair occurs in a known manner according to a natural growth rhythm including a growth phase (anagen), a transition phase (catagen) and a resting phase (telogen). A normal natural hair cycle begins when a hair bottom end is ejected or cast away at the beginning of the anagen phase. Consequently from 80 to 100 hairs fall out daily from the scalp. This number is an approximate value, which can occur over a shorter time and also can be greater or smaller, without causing fear regarding natural hair growth. However when the amount of hair falling out daily exceeds this approximate normal amount, an irreversible or reversible hair loss is occurring.

Androgenic (male) hair loss is the most common type of hair loss and is typically genetically determined. It is caused by increased sensitivity to Dihydrotestosterone (DHT) in certain parts of the scalp, and is passed on from generation to generation. DHT is converted from the male sex hormone Testosteron by the enzyme 5-α-reductase. Receptors exist on hair follicle cells that bind DHT. After binding to the receptor, DHT goes into the cell and interacts with the nucleus of the cell altering the production of hair protein (keratin) by the DNA in the nucleus of the cell. Ultimately growth of the hair follicle ceases. The hair growth cycle is affected in that the percentage of hairs in the growth phase (anagen) and the duration of the growth phase diminish resulting in shorter hairs. More hairs are in the resting state (telogen) and these hairs are much more subject to loss with the daily trauma of combing and washing. The hair shafts in male pattern baldness become progressively miniaturized, smaller in diameter and length, with time. In men with male pattern baldness all the hairs in an affected area may eventually (but not necessarily) become involved in the process and may with time cover the region with fine (vellus) hair. Pigment (color) production is also terminated with miniaturization so the fine hair becomes lighter in color. The lighter color, miniaturized hairs cause the area to first appear thin.

The condition usually starts in men aged 20 to 30 and follows a typical pattern. First, a receding hairline develops, and gradually the hair on top of the head also begins to thin. Eventually, the two balding areas meet to form a typical U-shape around the back and sides of the head.

Besides genetic predisposition, hair loss can also occur after feverish and consumptive illnesses, as a result of an unbalanced diet, because of dietary iron deficiency, due to albumin-free diet, during cures involving fasting and as a result of taking medicine to treat cancer. Hair loss also occurs in women after a birth. Gestagen, the hormone, which is formed during the pregnancy in a great amount, synchronizes the growth cycle of the hair on the head. A very high ratio of anagen hair to telogen hair is the result. After the birth this ratio changes in the opposite manner, which means many anagen hairs change into telogen hairs. After several months a shift into the anagen phase occurs during which the hairs can fall out simultaneously leading to a conspicuous bare spot in the hair on the head. When the new anagen hairs have reached their original length, the original state is again reached. Hair loss after massive psychic stress is another form of reversible hair loss. How this type of hair loss occurs is unknown. It appears that stress, which can impair many functions of our metabolism, also affects the operation of the hair organ running continuously at maximum performance

Therefore, a need exists for care products suitable for reducing or preventing hair loss, promoting the growth of hair, strengthen the hair and/or accelerating the growth of hair. In particular, there is a need for such products which can be administered topically.

EP0116439A2 discloses a hair tonic composition, comprising an an effective ingredient at least one of specified C10-C20 fatty acid, or a salt thereof. The hair tonic composition prevents the generation of dandruff in hair and itching in the scalp, and is said to accelerate the growth of hair.

US2002/0034485A1 discloses a hair tonic preventing or at least reducing hair loss, which hair tonic includes at least one saturated and unsaturated C10-C18 fatty acid, or a salt thereof, biotin and/or caffeine.

US2019/0216696A1 discloses the use of specified derivates of sandal pentanol for promoting hair growth and/or inhibiting or delaying hair loss in the human scalp.

WO2020/069522A1 discloses a cosmetic formulation comprising an effective amount of sandal pentanol or analogs thereof, and minoxidil or rosemary oil or emu oil or a combination thereof for the treatment and regrowth of mammalian hair loss.

### SUMMARY OF THE INVENTION

According to the present invention, it has now surprisingly been found that compositions containing a combination of the active ingredients sandal pentanol, caffeine, and C10-C18 fatty acid(s) is effective in promoting hair growth, and/or reducing or preventing hair loss. The combination of active ingredients in the compositions according to the present invention were found more effective than these active ingredients individually.

According to an aspect, subject matter of the present invention is a composition for promoting natural hair growth and/or reducing hair loss, in particular of mammals such as humans. The composition comprises sandal pentanol, caffeine, one or more saturated and unsaturated C10-C18 fatty acids, and a solvent.

The composition may further comprise one or more components that provide for a beneficial effect (denoted herein as “supportive components”), one or more oil components, one or more mineral components, and one or more cosmetically acceptable additives.

According to embodiments, the composition is a hair tonic comprising a water-alcohol-based solvent system.

According to other embodiments, the composition is a dry shampoo, in particular a dry shampoo to be applied in the form of an aerosol.

According to embodiments, the composition is a ready-to-use scalp-and-hair treatment product selected from hair tonic, hair lotion, hair conditioner, hair cream, hair shampoo, dry shampoo, hair rinse, hair liquid, hair oil, hair pomade, hair foam, hair spray.

According to another aspect, subject matter of the present invention is a hair tonic comprising sandal pentanol, caffeine, and C10-C18 fatty acid(s) in specified amounts, and in combination with specified components and/or optional components.

According to still another aspect, subject matter of the present invention is a hair tonic consisting essentially of sandal pentanol, caffeine, and C10-C18 fatty acid(s) in specified amounts, and in combination with specified components and/or optional components.

According to yet another aspect, subject matter of the present invention is a method for reducing or treating hair loss, which method comprises applying the composition or hair tonic according to the subject invention to scalp.

According to a further aspect, subject matter of the present invention is the use of the composition or hair tonic according to the subject invention for promoting natural mammalian hair growth and/or reducing mammalian hair loss.

According to a still further aspect, subject matter of the present invention is the composition or hair tonic according to the subject invention for use in the treatment of alopecia in mammals.

The compositions of the present invention are suitable for promoting hair growth, and/or inhibiting or delaying hair loss. The compositions further are suitable for topical administration on the human scalp. The compositions of the present invention are suitable both for cosmetic and therapeutic use in promoting hair growth and/or the treatment of hair loss in the human scalp.

When the active ingredient combination according to the present disclosure is applied to the scalp, the activity of the enzyme 5-alpha-reductase may be reduced or inhibited so that less 5-alpha-dihydrotestosterone responsible for human hair loss is formed. Also the blood supply in the application area may be increased as well as the flow of blood, and thus the nutrient supply in the hair organ. The keratinization, the tear resistance, the strength of the hair and thus the resistance of the hair to environmental influences and treatments may be increased. The anchoring of the hair bottom end in the scalp may be strengthened. The number of lost hairs may decrease, and hair loss be stabilized at a low level.

### DEFINITIONS

In the specification that follows , the terms “sandal pentanol” and “Sandalore” are used interchangeably. Sandal pentanol is a synthetic odorant having a fragrance similar to sandalwood, and which consequently is used in perfumes, emollients and skin cleaning agents as a less expensive ingredient mimicking sandalwood scent. Sandal pentanol, as well as structurally similar analogs thereof such as the compound named brahmanol, are alcohols having a chemical structure quite distinct from the ingredients of natural sandalwood oil, which are extracted by steam distillation from matured sandalwood trees, and are well-known valued components for perfumes. The IUPAC denomination of sandal pentanol is 3-methyl-5-(2,2,3-trimethylcyclopent-3-en-1-yl)pentan-2-ol.

As used in the specification and the appended claims, the singular forms “a,” “an” and “the” include plural referents unless the context clearly dictates otherwise.

The term “may” in the context of this application means “is permitted to” or “is able to” and is a synonym for the term “can.” The term “may” as used herein does not mean possibility or chance.

The term “and/or” in the context of this application means one or the other or both. For example, an aqueous solution of A and/or B means an aqueous solution of A alone, an aqueous solution of B alone and an aqueous solution of a combination of A and B.

The term “about” is understood to mean±10 percent of the recited number, numbers or range of numbers. According to embodiments, the term “about” is understood to mean ±2 percent of the recited number, numbers or range of numbers.

As used herein, the term “optionally” means that the corresponding step or feature may or may not be present. It includes both possibilities.

The terms “parts-by-weight” (PBW) and “parts-by-volume” (PBV) are used in their usual meaning. When used in a mixed context, for example an amount of a component indicated in PBW in admixture with a component indicated in PBV, the terms PBW and PBV as used herein denote the amount in grams of the component indicated in PBW relative to a volume in milliliters of the amount of the component indicated in PBV.

The term “composition” as used herein denotes a mixture or blend comprising at least two components. Typical compositions comprise at least one functional component (such as, for example, an active ingredient such as sandal pentanol) and at least one solvent component (such as water or an alcohol). A composition may be present in any suitable physical form, including for example liquids of varying viscosities, gels, foams, creams, and the like. The term “composition” may denote a ready-to-use product unless the context dictates otherwise.

“Ready-to-use scalp-and-hair treatment product ” denotes the form in which the composition is actually applied to human scalp, and hair.

The term “essentially free” of a particular component denotes an amount of less than 1.0% by weight of the respective component in an entity such as a composition, based on the total weight of the entity. In particular, the term “essentially free” denotes an amount of less than 0.5% by weight, for example less than 0.1% by weight, such as less than 0.01% by weight of the respective component in the entity.

The term “free” of a particular component denotes an amount below the detection limit, assuming that detectability is on a ppm (parts per million) basis (by weight). In particular, the term “free” of a particular component denotes an amount below 50 ppm by weight, for example less than 10 ppm by weight, such as less than 5 ppm by weight of the respective component in the entity.

The term “consisting essentially of” denotes the absence of additional, not specifically recited components in an entity such as a composition in an amount of more than 1.0% by weight, based on the total weight of the entity. In particular, the term “consisting essentially of” denotes the absence of additional, not specifically recited components in an amount of more than 0.5% by weight, for example more than 0.1% by weight, such as more than 0.01% by weight in an entity. It goes without saying that any additional, not specifically recited component is compatible with the specifically recited components and does not interfere with the function thereof.

The term “consisting of” denotes the absence of additional, not specifically recited components in an entity such as a composition in an amount amount below the detection limit, assuming that detectability is on a ppm (parts per million) basis (by weight). In particular, the term “consisting of” denotes the absence of additional, not specifically recited components in an amount of more than 50 ppm by weight, for example more than 10 ppm by weight, such as more than 10 ppm by weight in an entity. It goes without saying that any additional, not specifically recited component is compatible with the specifically recited components and does not interfere with the function thereof.

### DETAILED DESCRIPTION OF THE INVENTION

The inventors of the present invention surprisingly found that compositions containing a combination of the active ingredients sandal pentanol, caffeine, and C10-C18 fatty acid(s) are effective in a synergistic manner for promoting hair growth, and/or reducing or preventing hair loss.

According to an aspect, subject matter of the present disclosure is a composition for promoting natural mammalian hair growth and/or reducing mammalian hair loss. The composition comprises sandal pentanol, caffeine, one or more saturated and unsaturated C10-C18 fatty acids, and a solvent.

### 1 The Active Ingredient Combination

The active ingredients of the compositions according to the present disclosure comprise a combination of:
sandal pentanol, also denoted as Sandalore,
caffeine,
one or more saturated and/or unsaturated C10-C18 fatty acids.

Sandal pentanol is the first active ingredient of the active ingredient combination used according to the present disclosure. Sandal pentanol is a synthetic odorant having a fragrance used as an ingredient mimicking the sandalwood scent. Sandal pentanol is an alcohol having a chemical structure quite distinct from the ingredients of natural sandalwood oil which usually are extracted by steam distillation of wood from matured sandalwood trees. The IUPAC denomination of sandal pentanol is 3-methyl-5-(2,2,3-trimethylcyclopent-3-en-1-yl)pentan-2-ol. Sandal pentanol has been shown to have a stimulatory effect on the olfactory receptor OR2AT4, which is expressed in the epithelium of human hair follicles. Simulation of OR2AT4 by sandal pentanol in turn prolongs hair growth ex vivo by decreasing apoptosis and increasing the production of growth factor IGF-1. Sandal pentanol accordingly in believed to act as an agonist promoting the growth of mammalian hair.

Analogs or derivatives, respectively, of sandal pentanol are believed to have a similar effect on mammalian hair growth, and may be used in the present disclosure alternatively or additionally to sandal pentanol. Sandal pentanol analogs suitable as active ingredients include:
(4Z-3-methyl-5-(2,2,3-trimethylcyclopent-3-en-1-yl)pent-4-en-2-ol (sandal pentenol)
1-methyl-2-((1,2.2-trimethylbicyclo(3.1.0)hex-3-yl)methyl)-cyclopropane-methanol (sandal cyclopropane)
(E)-3,3-dimethyl-5-(2,2,3-trimethylcyclopent-3-en-1-yl)pent-4-en-2-ol (santol pentenol)
2-methyl-4-(2,2,3-trimethyl-3-cyclopenten-1-yl)butanol (sandal cyclopentane)
(E)-2-ethyl-4-(2,2,3-trimethylcyclopent-3-en-1-yl)but-2-en-1-ol (sandalrome)
(E)-2-methyl-4-(2,2,3-trimethyl-3-cyclopenten-1-yl)-2-buten-1-ol (sandal butenol)

According to embodiments, the compositions according to the present disclosure comprise one or more of the above sandal pentanol analogs in addition to the active ingredient sandal pentanol. If present, sandal pentanol plus the one or more sandal pentanol analogs are present in total amounts as indicated below for sandal pentanol alone.

According to other embodiments, the compositions according to the present disclosure comprise sandal pentanol as an active ingredient, and are essentially free of, or free of the above sandal pentanol analogs.

According to further, less preferred embodiments, the compositions according to the present disclosure comprise one or more of the above sandal pentanol analogs substituting for the active ingredient sandal pentanol.

Caffeine is the second active ingredient of the active ingredient combination used according to the present disclosure. Caffeine is a plant alkaloid also known under the names theine, methyltheobromine and guaranine. The IUPAC denomination is 1,3,7-trimethylxanthine. Caffeine is a well-known stimulant which is present for example in coffee, black tea, but also in cocoa solids and products containing same (in particular dark chocolate), or cola nuts. Caffeine has a vasodilating effect, thereby increasing blood and nutrients supply to the hair roots. Studies have shown that caffeine also enhances the hair shaft elongation (making the hair root bigger), prolonges anagen duration (increasing the hair prowth phase) and stimulates the proliferation of hair matrix keratinocytes (increasing keratin production).

The third active ingredient of the active ingredient combination used according to the present disclosure are C10-C18 fatty acids. Studies suggest that fatty acids may have a hair-growth promoting effect, and at least some fatty acids may have an inhibitory effect on the enzyme testosterone-5-alpha-reductase. This enzyme catalyzes the conversion of testosterone to 5-alpha-dihydrotestosterone (5α-DHT, or simply DHT). Studies have shown that DHT is a major cause for hair loss both in males and females.

The fatty acid(s) used in the active ingredient combination is/are one or more linear and/or branched, saturated and/or unsaturated C10-C18 fatty acids. Typically, the one or more C10-C18 fatty acids will be linear fatty acids. According to embodiments, the C10-C18 fatty acid(s) may be selected from capric acid (C10:0), lauric acid (C12:0), myristic acid (C14:0), isomyristic acid (C14:0), palmitic acid (C16:0), palmitoleic acid (C16:1), stearic acid (C18:0), oleic acid (C18:1), linoleic acid (C18:2), linolenic acid (C18:3), parinaric acid (C18:4), petroselinic acid (C18:1), or combinations thereof.

According to embodiments, the C10-C18 fatty acid(s) may comprise lauric acid (C12:0), myristic acid (C14:0), or a combination thereof. According to specific embodiments, the C10-C18 fatty acid(s) may be selected from lauric acid (C12:0), myristic acid (C14:0), or a combination thereof. For example, the fatty acid component of the active ingredient combination may be lauric acid (C12:0).

According to embodiments, the fatty acid component of the active ingredient combination may comprise one or more unsaturated fatty acids. It is believed that at least some unsaturated fatty acids may have a higher inhibitory effect on testosterone-5-alpha-reductase than saturated fatty acids. According to embodiments, the C10-C18 fatty acid(s) may comprise palmitoleic acid (C16:1), oleic acid (C18:1), linoleic acid (C18:2), linolenic acid (C18:3), parinaric acid (C18:4), petroselinic acid (C18:1), or a combination thereof. For example, the fatty acid component of the active ingredient combination may comprise linoleic acid (C18:2).

According to specific embodiments, the fatty acid component of the active ingredient combination may be selected from palmitoleic acid (C16:1), oleic acid (C18:1), linoleic acid (C18:2), linolenic acid (C18:3), parinaric acid (C18:4), petroselinic acid (C18:1), or a combination thereof. For example, the fatty acid component of the active ingredient combination may be selected from palmitoleic acid (C16:1) and/or linoleic acid (C18:2).

If the fatty acid component of the active ingredient combination comprises one or more unsaturated fatty acids, the respective compositions preferably comprise an antioxidant in order to reduce autoxidation of the double bonds.

According to embodiments, the composition according to the present disclosure may be essentially free, or free of unsaturated C10-C18 fatty acids. According to specific embodiments, the composition according to the present disclosure may be essentially free, or free of unsaturated fatty acids per se.

The C10-C18 fatty acid(s) may be present in the form of free acid, or in the form of a cosmetically acceptable salt, for example in the form of an alkali metal salt, alkaline earth metal salt, ammonium salt or combination thereof. According to particular embodiments, the C10-C18 fatty acid(s) may be present in the form of the free acid(s).

### 2 Amounts of Active Ingredients

The active ingredients per se, and the combination thereof in the compositions according to the present disclosure are present in amounts effective to provide for the hair loss-reducing and/or hair growth-promoting effect according to the invention.

The amount of sandal pentanol in the compositions according to the present disclosure is not particularly critical. Typically, the sandal pentanol will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. According to embodiments, the sandal pentanol may be present in an amount of 0.050 to 5.0 percent-by-weight, based on the weight of the composition. According to more specific embodiments, the sandal pentanol may be present in an amount of 0.10 to 2.0 percent-by-weight, based on the weight of the composition. According to even more specific embodiments, the sandal pentanol may be present in an amount of 0.50 to 1.5 percent-by-weight, based on the weight of the composition.

If analogs of sandanol pentanol are present, either replacing at least in part the sandal pentanol, or in addition to sandal pentanol, the total amount of sandal pentanol plus one or more of sandal pentanol analogs (as indicated above) is within the ranges indicated above for sandal pentanol exclusively.

The amount of caffeine in the compositions according to the present disclosure is not particularly critical. Typically, the caffeine will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. According to embodiments, the caffeine may be present in an amount of 0.050 to 5.0 percent-by-weight, based on the weight of the composition. According to more specific embodiments, the caffeine may be present in an amount of 0.10 to 2.0 percent-by-weight, based on the weight of the composition. According to even more specific embodiments, the caffeine may be present in an amount of 0.50 to 1.5 percent-by-weight, based on the weight of the composition.

Similarly, the amount of C10-C18 fatty acid(s) in the compositions according to the present disclosure is not particularly critical. Typically, the C10-C18 fatty acid(s) will be present in an amount of from 0.010 to 10.0 percent-by-weight, based on the weight of the composition. If the amount is below 0.010 percent-by-weight, the effect sought by the present disclosure may be difficult to achieve. Amounts above 10.0 percent-by-weight may not further enhance the effect, but contribute to higher costs of the material resources for the compositions according to the present disclosure. High amounts of C10-C18 fatty acid(s) further may require higher amounts of surfactants, and may result in a greasy appearance or hair feel. According to embodiments, the C10-C18 fatty acid(s) may be present in an amount of 0.050 to 5.0 percent-by-weight, based on the weight of the composition. According to more specific embodiments, the C10-C18 fatty acids may be present in an amount of 0.10 to 2.0 percent-by-weight, based on the weight of the composition. According to even more specific embodiments, the C10-C18 fatty acids may be present in an amount of 0.50 to 1.5 percent-by-weight, based on the weight of the composition.

The combination of the active ingredients in the compositions according to the present disclosure as well is not particularly critical. The amounts of the active ingredient combination are selected in order to provide for the effect sought. Typically, the sandal pentanol will be present in an amount of from 0.010 to 10.0 percent-by-weight, the caffeine will be present in an amount of from 0.010 to 10.0 percent-by-weight, and the one or more C10-C18 fatty acid(s) will be present in an amount of from 0.010 to 2.0 percent-by-weight, each based on the weight of the composition. Conveniently, the sandal pentanol will be present in an amount of from 0.10 to 5.0 percent-by-weight, the caffeine will be present in an amount of from 0.10 to 5.0 percent-by-weight, and the one or more C10-C18 fatty acid(s) will be present in an amount of from 0.050 to 3.0 percent-by-weight, each based on the weight of the composition.

According to embodiments, the sandal pentanol will be present in an amount of from 0.50 to 2.0 percent-by-weight, the caffeine will be present in an amount of from 0.50 to 2.0 percent-by-weight, and the one or more C10-C18 fatty acid(s) will be present in an amount of from 0.10 to 2.0 percent-by-weight, each based on the weight of the composition. According to more specific embodiments, the sandal pentanol will be present in an amount of from 0.80 to 1.50 percent-by-weight, the caffeine will be present in an amount of from to 1.50 percent-by-weight, and the one or more C10-C18 fatty acid(s) will be present in an amount of from to 1.0 percent-by-weight, each based on the weight of the composition.

### 3 Optional Added-Benefit Components

The compositions according to the present disclosure may optionally comprise additional components which enhance, supplement or add to the benefit obtained by the active ingredient combination. Such additional optional components will be referred to in the following as supportive component, oil component and mineral component. The supportive component covers a broad spectrum of substances having diversified influences on physiologic or metabolic processes. The oil component covers natural essential oils and modified oils. Modified oils include in particular hydrogenated oils and oil constituents. The mineral component covers minerals in water-soluble form.

According to embodiments, the compositions according to the present disclosure further may comprise a supportive component. Examples of such supportive components include in particular vasodilatators, blood circulation stimulators, antioxidants, vitamins, provitamins. If present, the amount of the supportive component or combinations thereof will be in the range of from 0.0010 to 5.0, in particular from to 3.0 percent-by-weight, based on the weight of the composition.

The supportive component may comprise, for example, one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol (provitamin B5), omega-3 fatty acids, omega-6 fatty acids, folates, cannabidiol, saw palmetto, minoxidil, or combinations thereof.

According to embodiments, the supportive component includes a vasodilator selected from tocopherols (alpha-, beta-, gamma- or delta-tocopherol), in particular alpha-tocopherol, tocopherol esters (including, for example, tocopherol acetate, tocopheryl succinate, tocopherylnicotinate, tocopherylpoly(oxyethylene)-succinate), nicotinic acid, nicotinic amides, nicotinic esters, camphor; an antioxidant such as vitamin C or polyphenol(s); an inhibitor of testosterone-5-alpha-reductase such as the omega-6 fatty acid arachidonic acid; a substance providing a benefit to hair health such as nicotinic amide (niacinamide); or a combination thereof. According to embodiments, the supportive component includes tocopherol or tocopheryl acetate (vitamin E acetate), and/or niacinamide. Suitable vasodilatory plant extracts include burned or distilled nettle extract (Urtica Diocia), roast chestnut extract (Aesculus Hippocastanum), Arnica extract (Arnica Montana), or hemp extract.

The oil component may comprise, for example, peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof. Such oil components may — inter alia — promote hair growth, contribute to scalp health, or strenghten the hair. If present, the oil component will be present in the compositions of the present disclosure in an amount of from 0.0010 to 1.0, in particular from 0.010 to 0.50 percent-by-weight, based on the weight of the composition.

According to embodiments, the oil component includes peppermint oil and/or hydrogenated ethylhexyl olivate.

The mineral component includes metal ions having a beneficial effect on hair growth and/or scalp health. The mineral component may comprise, for example, iron, zinc, or a combination thereof. If present, the mineral component will be present in the compositions of the present disclosure in 0.0050-0.50 molar concentration, for example in 0.010-0.25 molar concentration.

The compositions according to the present disclosure may comprise a supportive component, an oil component or a mineral component alone or in any combination.

### 4 The Solvent or Carrier System, and pH Range

The solvent system used in the compositions of the present disclosure is water-based, alcohol-based or water-alcohol-based. Typically, the solvent comprises water and one or more monovalent and/or polyvalent C1-C4 alcohols. According to embodiments, the solvent comprises water and an alcohol selected from ethanol, n-propanol, isopropanol, glycerol, ethylene glycol, propylene glycol or a combination thereof.

The solvent further may comprise one or more solubilisation enhancers. The solubilisation enhancers obviously are intended to solubilize compounds which are not or only partially soluble in the solvent at the concentration used in the compositions. Solubilisation enhancers generally include surfactants. Surfactants include anionic, cationic, amphoteric and non-ionic surfactants.

The anionic surfactants may be selected from the group consisting of salts (such as alkaline salts, for example, sodium salts, ammonium salts, amine salts, amino alcohol salts and magnesium salts) of the following compounds: alkyl sulphates, alkyl ether sulphates, alkylamido ether sulphates, alkylarylpolyether sulphates, monoglyceride sulphates; alkyl sulphonates, alkyl phosphates, alkylamide sulphonates, alkylaryl sulphonates, alpha-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates; N-acyltaurates; and mixtures thereof. The alkyl or acyl radical of all of these various compounds, for example, comprises from 8 to 24 carbon atoms, and the aryl radical, for example, is chosen from phenyl and benzyl groups. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof. Anionic derivatives of polysaccharides, for example carboxyalkyl ether of alkyl polyglucosides, can be also used.

Suitable anionic surfactant(s) may comprise at least one anionic functional groups at their head selected from sulfate, sulfonate, phosphate and carboxylates. In order to avoid any overlap with the active ingredient combination of the present disclosure, anionic surfactant(s) for use in the compositions according to the present disclosure exclude fatty acids or salts thereof.

Suitable alkyl sulfates include ammonium lauryl sulfate, sodium lauryl sulfate (sodium dodecyl sulfate, SLS, or SDS), and alkyl-ether sulfates, such as sodium laureth sulfate (sodium lauryl ether sulfate or SLES), and sodium myreth sulfate.

Further suitable anionic surfactants may include docusate (dioctyl sodium sulfosuccinate), alkyl-aryl ether phosphate, alkyl ether phosphate, sodium lauroyl sarcosinate, ammonium laureth sulfate, disodium lauryl sulfosuccinate, and sodium lauryl sulphoacetate.

Preferred anionic surfactants may be selected from the group consisting of sodium laurylethersulfate, sodium laurethethersulfate, sodium dodecyl sulfate, ammonium laurethethersulfat, ammonium dodecyl sulfate, alkylbenzenesulfonate, and combinations thereof.

The surfactant may be a non-ionic surfactant. The non-ionic surfactant may be selected from the group consisting of lanolin alcohol, and polyoxyethylene ethers of fatty alcohols, and mixtures thereof. The non-ionic surfactant may be preferably ceteareth-n, wherein n is from 2 to 100, or from 10 to 30. Suitable nonionic surfactants are compounds that are well known (see, for example, in this respect “Handbook of Surfactants” by M. R. Porter, published by Blackie & Son (Glasgow and London), 1991, pp. 116-178).

Preferred surfactants encompass non-ionic surfactants, in particular polyoxyethylated fatty alcohols or oils. Particularly preferred non-ionic surfactants include polyoxyethylated Castor Oil and hydrogenated polyoxylated Castor Oil. Specific examples of non-ionic surfactants that may be used in the compositions of the present disclosure are PEG-35 Castor Oil, PEG-40 Hydrogenated Castor Oil, and combinations thereof.

The compositions of the present disclosure can have a pH within the range of from 3.0 to 8.0. According to embodiments, the pH may be within the range of from 4.0 to 6.5, for example within the range of from 4.5 to 6.0.

### 5 Optional Cosmetically Acceptable Additives

The compositions according to the present disclosure optionally may comprise cosmetically acceptable additives, which may contribute to achieve or enhance the properties or effects sought. According to embodiments, the cosmetically acceptable additives may be selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents. The cosmetically acceptable additives may be used in any combinations, as desired or required.

Suitable cosmetically acceptable additives not specifically described below are listed in the International Cosmetics Ingredient Dictionary and Handbook, (8th ed.; The Cosmetics, Toiletry, and Fragrance Association). Particularly, vol. 2, sections 3 (Chemical Classes) and 4 (Functions), which are useful in identifying specific additives to achieve a particular purpose or multipurpose. A few of these additives are discussed below, whose disclosure is of course non-exhaustive.

Suitable amount ranges for perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents are indicated for additives discussed below. These cosmetically acceptable additives may be present in a total amount of up to 20.0, in particular up to 15.0 percent-by-weight, based on the weight of the composition. Typically, the total amount of these cosmetically acceptable additives may be within a range of from 0.010 to 10.0, conveniently within a range of from 0.010 to 7.0 percent-by-weight, based on the weight of the composition. For example, the total amount of these cosmetically acceptable additives may be within a range of from 0.010 to 5.0, conveniently within a range of from 0.010 to 3.0 percent-by-weight, based on the weight of the composition.

### 5.1 Perfume or Fragrance

The compositions according to the present disclosure may comprise a perfume or fragrance. For example, the compositions may comprise from 0.0010 to 1.50, in particular 0.010-0.50 percent-by-weight perfume, based on the weight of the composition. Perfume can provide an enhanced user experience by making the composition smell pleasant and/or invoke emotions, such as relaxing or exciting smells.

Alternatively, compositions according to the present disclosure may be substantially free of perfume and/or fragrance. Some consumers prefer perfume-free compositions.

The perfume may be an animal fragrance or a plant fragrance. The animal fragrance may be selected from the group consisting of musk oil, civet, castoreum, ambergris, and mixtures thereof.

The plant fragrance may be selected from the group consisting of nutmeg extract, cardomon extract, ginger extract, cinnamon extract, patchouli oil, geranium oil, orange oil, mandarin oil, orange flower extract, cedarwood, vetyver, lavandin, ylang extract, tuberose extract, sandalwood oil, bergamot oil, rosemary oil, spearmint oil, peppermint oil, lemon oil, lavender oil, citronella oil, chamomille oil, clove oil, sage oil, neroli oil, labdanum oil, eucalyptus oil, verbena oil, mimosa extract, narcissus extract, carrot seed extract, jasmine extract, olibanum extract, rose extract, and mixtures thereof.

The perfume may comprise one or more scents selected from the group consisting of acetophenone, adoxal, aldehyde C-12, aldehyde C-14, aldehyde C-18, allyl caprylate, ambroxan, amyl acetate, dimethylindane derivatives, α-amylcinnamic aldehyde, anethole, anisaldehyde, benzaldehyde, benzyl acetate, benzyl alcohol and ester derivatives, benzyl propionate, benzyl salicylate, borneol, butyl acetate, camphor, carbitol, cinnamaldehyde, cinnamyl acetate, cinnamyl alcohol, cis-3-hexanol and ester derivatives, cis-3-hexenyl methyl carbonate, citral, citronnellol and ester derivatives, cumin aldehyde, cyclamen aldehyde, cyclo galbanate, damascones, decalactone, decanol, estragole, dihydromyrcenol, dimethyl benzyl carbinol, 6,8-dimethyl-2-nonanol, dimethyl benzyl carbinyl butyrate, ethyl acetate, ethyl isobutyrate, ethyl butyrate, ethyl propionate, ethyl caprylate, ethyl cinnamate, ethyl hexanoate, ethyl valerate, ethyl vanillin, eugenol, exaltolide, fenchone, fruity esters such as ethyl 2-methyl butyrate, galaxolide, geraniol and ester derivatives, helional, 2-heptonone, hexenol, α-hexylcinnamic aldehyde, hydroxycitrolnellal, indole, isoamyl acetate, isoeugenol acetate, ionones, isoeugenol, isoamyl iso-valerate, iso E super, limonene, linalool, lilial, linalyl acetate, lyral, majantol, mayol, melonal, menthol, p-methylacetophenone, methyl anthranilate, methyl cedrylone, methyl dihydrojasmonate, methyl eugenol, methyl ionone, methyl-α-naphthyl ketone, methylphenylcarbinyl acetate, mugetanol, γ-nonalactone, octanal, phenyl ethyl acetate, phenyl-acetaldehyde dimethyl acetate, phenoxyethyl isobutyrate, phenyl ethyl alcohol, pinenes, sandalore, santalol, stemone, thymol, terpenes, triplal, triethyl citrate, 3,3,5-trimethylcyclohexanol, γ-undecalactone, undecenal, vanillin, veloutone, verdox, and mixtures thereof.

### 5.2 Opacifiers or Turbidity-Inducing Agents

The compositions in accordance with the present disclosure may comprise an opacifier, which sometimes is denoted as well by the term “turbidity-inducing agent”. For example, the compositions may comprise from 0.10 to 5.0, in particular 0.50-2.0 percent-by-weight opacifier, based on the weight of the composition. Suitable opacifiers include mineral pigments such as, for example, titanium dioxide, alumina, calcium carbonate. Other suitable opacifiers include fatty alcohols and fatty acid esters such as, for example, cetearyl alcohol, cetyl alcohol, ethylene glycol distearate, glyceryl stearate, glyceryl oleate, stearyl stearate.

### 5.3 Preservatives

The compositions in accordance with the present disclosure may comprise a preservative or mixture of preservatives. The compositions may comprise the preservative(s) in an amount of up to 1.0, for example from 0.0010 to 1.0, in particular from 0.010 to 0.50 percent-by-weight, based on the weight of the composition. Cosmetically acceptable preservatives include organic acids such as para-hydroxybenzoic acid; organic acid salts such as sodium benzoate; compounds such as benzyl alcohol, phenoxyethanol, 1,3-bis(hydroxymethyl)-5,5-dimethylimidazolidine-2,4-dione; and mixtures thereof.

### 5.4 pH Modifiers

The compositions in accordance with the present disclosure may comprise a pH modifier and/or buffering agent, in a sufficient amount to effectively adjust the pH of the composition. For example, the compositions may comprise up to 1.0, for example from 0.000010 to 1.0, in particular 0.0010-0.50 percent-by-weight pH modifiers, based on the weight of the composition. Suitable pH modifiers and/or buffering agents for use herein include, but are not limited to: ammonia, alkanolamines such as monoethanolamine, diethanolamine, triethanolamine, monopropanolamine, dipropanolamine, tripropanolamine, tripropanolamine, 2-amino-2-methyl-1-propanol, and 2-amino-2-hydroxymethyl-1,3,-propandiol and guanidium salts, alkali metal and ammonium hydroxides and carbonates, such as sodium hydroxide, sodium silicate, sodium meta silicate and ammonium carbonate, and acids such as organic and inorganic acids, for example ascorbic acid, citric acid, acetic acid or tartaric acid, phosphoric acid, hydrochloric acid, and mixtures thereof.

Preferred alkaline pH modifiers include ammonia, ethanolamines, and alkali metal hydroxides, in particular sodium hydroxide. Preferred acidic pH modifiers include hydrochloric acid, ascorbic acid, or citric acid.

### 5.5 Chelators

The compositions according to the present disclosure may further comprise one or more chelators (also known as “chelating agent”, “sequestering agent”, or “sequestrant”) in an amount sufficient to reduce the amount of metals available to interact with formulation components. Chelators are well known in the art and a non-exhaustive list thereof can be found in A E Martell & R M Smith, Critical Stability Constants, Vol. 1, Plenum Press, New York & London (1974) and A E Martell & R D Hancock, Metal Complexes in Aqueous Solution, Plenum Press, New York & London (1996). Typically, the compositions may comprise one or more chelators in a total amount of from 0.0010 to 1.0, in particular from 0.010 to 0.50 percent-by-weight, based on the weight of the composition.

The one or more chelators may be selected from the group consisting of carboxylic acids (such as aminocarboxylic acids), phosphonic acids (such as aminophosphonic acids), polyphosphoric acids (such as linear polyphosphoric acids), their salts thereof, and mixtures thereof.

By “salts thereof”, it is meant—in the context of chelators—all salts comprising the same functional structure as the chelators they are referring to and including alkali metal salts, alkaline earth salts, ammonium salts, substituted ammonium salts, and mixtures thereof; alternatively sodium salts, potassium salts, calcium salts, magnesium salts, ammonium salts, and mixtures thereof; alternatively monoethanolammonium salts, diethanolammonium salts, triethanolammonium salts, and mixtures thereof.

The one or more chelators may be one or more aminocarboxylic acid chelators comprising one or more carboxylic acid moieties (—COOH) and one or more nitrogen atoms. The one or more aminocarboxylic acid chelators may be selected from the group consisting of diethylenetriamine pentaacetic acid (DTPA), ethylenediamine disuccinic acid (EDDS), ethylenediamine-N,N′-diglutaric acid (EDDG), 2-hydroxypropylenediamine-N-N′-disuccinic acid (HPDDS), glycinamide-N,N′-disuccinic acid (GADS), ethylenediamine-N-N′-diglutaric acid (EDDG), 2-hydroxypropylenediamine-N-N′-disuccinic acid (HPDDS), ethylenediaminetetraacetic acid (EDTA), ethylenedicysteic acid (EDC), ethylenediamine-N-N′-bis(ortho-hydroxyphenyl acetic acid) (EDDHA), diaminoalkyldi(sulfosuccinic acids) (DDS), N,N′-bis(2-hydroxybenzyl)ethylenediamine-N,N′-diacetic acid (HBED), their salts thereof, and mixtures thereof. Conveniently, a chelator used in compositions according to the present disclosure, if any, may be EDTA.

### 5.6 Viscosity Modifiers or Rheology Modifiers

The compositions in accordance with the present disclosure may comprise viscosity modifiers, referred to in the art as well as “thickening agents”. If present, such viscosity modifiers typicall will be used in the compositions in an amount of from 0.010 to 3.0, in particular from 0.50 to 1.0 percent-by-weight, based on the weight of the composition. Suitable cosmetically acceptable viscosity modifiers include non-ionic polymers, for example polysaccharides such as cellulose, cellulose gum or xanthan gum, and non-ionic viscosity modifiers based on polyether-1 such as the commercially available viscosity modifiers Pure Thix 1442.

### 5.7 Further Cosmetically Acceptable Additives

The compositions may further comprise care materials, such as hair-care providing or skin-care-providing plant or vegetable extracts, cationic polymers or cationic surfactants. Typical amounts of care materials in the compositions according to the present disclosure will be in the range of from 0.010 to 5.0, in particular 0.050-2.0 percent-by-weight, based on the weight of the composition.

Additional cosmetically acceptable aditives, which may be present in the compositions according to the present disclosure include:
physiologically compatible silicone derivative compounds, such as volatile or non-volatile silicone or high molecular weight siloxane polymers, such as for example Quaternium-80, typically in an amount of from 0.05 to 20 percent-by-weight, in particular from 0.10 to 5.0 percent-by-weight, based on the weight of the composition
light protecting agents, such as for example octylmethoxy cinnamate
anti-flaking agents, typically in an amount of about 0.01 to 2 percent-by-weight
hair luster-imparting agents, such as for example phenyltrimethicone,
combability improving agents, such as for example cationic surfactants such as Behentrimonium Chloride, or cationic polymers such as Polyquaternium-10
defatting agents
conditioning agents
moisturizing agents, such as for example sodium lactate, sodium PCA, glycine, fructose, urea, niacinamide, inositol
cooling agents, such as for example menthol, eucalptol, geraniol, linalool, isopulegol, cubedol, p-mentane-3,8-diols, hydroxy-citronellal as well as esters and derivatives thereof; ketones such as menthone and carvone; carboxamides, or evaporative cooling agents

The cosmetically acceptable additives may be used in the compositions according to the present disclosure in any combinations, as desired or required.

### 6 Substances Potentially Excluded

The compositions according to the present disclosure may be essentially free of, or be free of certain substances or classes of chemical compounds.

According to embodiments, the compositions according to the present disclosure may be essentially free of biotin. According to embodiments, the compositions according to the present disclosure may be free of biotin. According to particular embodiments, the concentration of biotin may be less than 5 ppm by weight.

According to embodiments, the compositions according to the present disclosure may be essentially free of quaternary ammonium compounds. According to embodiments, the compositions according to the present disclosure may be free of quaternary ammonium compounds. According to particular embodiments, the concentration of quaternary ammonium compounds may be less than 5 ppm by weight.

According to embodiments, the compositions according to the present disclosure may be essentially free of silicones. According to embodiments, the compositions according to the present disclosure may be free of silicones. According to particular embodiments, the concentration of silicones may be less than 5 ppm by weight.

According to embodiments, the compositions according to the present disclosure may be essentially free of quaternary ammonium compounds and silicones. According to embodiments, the compositions according to the present disclosure may be free of quaternary ammonium compounds and silicones. According to particular embodiments, the concentration of quaternary ammonium compounds and silicones may be less than 5 ppm by weight.

According to embodiments, the polymeric compounds present in the the compositions according to the present disclosure exclusively are film-forming polymers. In other words, according to embodiments, the compositions according to the present disclosure may be essentially free of non-film-forming polymers. According to embodiments, the compositions according to the present disclosure may be free of non-film-forming polymers. According to particular embodiments, the concentration of non-film-forming polymers may be less than 5 ppm by weight.

According to embodiments, the compositions according to the present disclosure may be essentially free of or be free of non-film-forming polymers selected from acrylates/Steareth-20 methacrylate copolymer, carbomer, polyacrylamide, hydroxyethylcellulose, Xanthan Gum and polymers having similar chemistry.

According to embodiments, the compositions according to the present disclosure may be essentially free of or be free of unsaturated fatty acids.

### 7 Specific Composition Embodiments

The following describes embodiments of compositions according to the present disclosure formulated as liquid formulation or aerosol.

### 7.1 Liquid Formulation Embodiments

According to embodiments, the compositions according to the present disclosure may be formulated as liquid formulations. The liquid formulation may have a conventional base for a cosmetic composition, for example an aqueous base, an alcoholic base or an aqueous-alcoholic base. The composition in the form of a liquid formulation may comprise (in addition to the active ingredient combination), for example, 70.0 to 99.5 percent-by-weight water-alcohol-based solvent. In particular, the composition in the form of a liquid formulation may comprise, for example, 90.0 to 99.0 percent-by-weight, for example 92.0 to 98.0 percent-by-weight, based on the weight of the composition, water-alcohol-based solvent.

The composition in the form of a liquid formulation may comprise, for example, water in an amount of at least 10 percent-by-weight, based on the weight of the composition, and alcohol in an amount of at least 20 percent-by-weight, based on the weight of the composition. The solvent may comprise, for example, lower univalent alcohols suitable for cosmetic purposes, such as ethanol, n-propanol and isopropanol, or multivalent alcohols, such as glycerol, ethylene glycol, propylene glycol, or a combination thereof. The composition in the form of a liquid formulation may further comprise, solubilisation enhancers such as for example non-ionic surfactants. The pH of the composition in the form of a liquid formulation may be within the range of from 3 to 7, in particular within the range of from 4 to 6.5, for example within the range of from 4.5 to 6.

According to embodiments, the composition in the form of a liquid formulation may be a hair tonic.

### 7.2 Aerosol Formulation Embodiments

According to embodiments, the compositions according to the present disclosure may be formulated as aerosol formulations. The compositions in the form of an aerosol formulation may comprise (in addition to the active ingredient combination), for example, 70.0 to 99.5 percent-by-weight, in particular 90.0 to 99.0 percent-by-weight, for example 92.0 to 98.0 percent-by-weight, based on the weight of the composition, of a blend comprising starch particles and an alcohol-based solvent.

The starch may be derived from any plant, for example potatoe, rize, corn, or tapioca. According to embodiments, the starch may be rice starch, tapioca starch, or a combination thereof. The starch particles may be hydrophobically modified starch particles, hydrophobically unmodified starch particles, or a combination thereof.

The alcohol-based solvent may comprise, for example, lower univalent alcohols suitable for cosmetic purposes, such as ethanol, n-propanol and isopropanol, or multivalent alcohols, such as glycerol, ethylene glycol, propylene glycol, or a combination thereof. According to particular embodiments, the alcohol(s) may be selected from ethanol and isopropanol.

The blend may further comprise a propellant, such as propane, isobutane, n-butane, cyclopropane, or a combination thereof. The propane may comprise halogenated hydrocarbone, which in view of environmental concerns is less preferred, however. The propellant is preferably miscible with the alcohol-based solvent.

The blend of the compositions in the form of an aerosol formulation may further comprise inorganic particles. Such inorganic particles act as a dispersing agent to suspend any dispersed solids or liquids within the composition. Including inorganic particles in the aerosol composition may separate the starch particles upon settling between uses, thereby preventing the creation of particle aggregates that are difficult to disrupt mechanically (by shaking). Suitable inorganic particles for the aerosol blends include silicas, clays, and mixtures thereof. For example, hydrophilic fumed silica particles having a total surface area of greater than 100 m2/g are suitable as inorganic particles for the aerosol blends.

According to embodiments, the blend may comprise, based on the weight of the blend, 5.0 to 12.0 percent-by-weight starch particles, 30.0 to 50.0 percent-by-weight alcohol-based solvent, 40.0 to 60.0 percent-by-weight propellant, and optionally 0.50 to 1.0 percent-by-weight inorganic particles. According to more specific embodiments, the blend may comprise, based on the weight of the blend, 7.0 to 10.0 percent-by-weight starch particles, 33.0 to 38.0 percent-by-weight alcohol-based solvent, 52.0 to 60.0 percent-by-weight propellant, and optionally 0.60 to 0.90 percent-by-weight inorganic particles.

According to embodiments, the composition in the form of an aerosol formulation may be a dry shampoo.

### 7.3 Scalp-and-Hair Treatment Product

According to embodiments, the compositions according to the present disclosure may be formulated as ready-to-use scalp-and-hair treatment products. For example, the compositions according to the present disclosure may be a ready-to-use scalp-and-hair treatment product selected from hair tonic, hair lotion, hair conditioner, hair cream, hair shampoo, dry shampoo, hair rinse, hair liquid, hair oil, hair pomade, hair foam, hair spray.

According to preferred embodiments, after application the compositions according to the present disclosure are left on the scalp for a time sufficient to trigger the hair-growth promoting or hair-loss reducing effect of the active ingredient combination according to the present disclosure. For example, the compositions may be left on the scalp for at least 5 minutes, for example at least 15 minutes. The compositions may be left on the scalp for at least 30 minutes, for example at least 60 minutes.

According to preferred embodiments, the ready-to-use scalp-and-hair treatment product is a leave-on product. Leave-on products are applied to hair and/or scalp, and are not subsequently actively removed.

### 8 Hair Tonic

According to specific embodiments, the composition according to the present disclosure is a hair tonic, comprising:
0.90 to 1.20 percent-by-weight Sandal Pentanol,
0.90 to 1.20 percent-by-weight caffeine,
0.19 to 0.25 percent-by-weight of a C10-C18 fatty acid or an alkai metal salt thereof, wherein the fatty acid is selected from lauric acid (C12:0), myristic acid (C14:0), or a combination thereof,
0.010 to 0.60 percent-by-weight solubilisation enhancer,
0.010 to 0.50 percent-by-weight fragrance,
20.0 to 80.0 percent-by-weight alcohol,
ad 100 percent-by-weight water.

According to embodiments, the C10-C18 fatty acid or alkali metal salt thereof may be lauric acid (C12:0) or sodium laurate. For example, the C10-C18 fatty acid may be lauric acid (C12:0) in form of the free acid.

According to embodiments, the solubilisation enhancer of the hair tonic may be selected from PEG-ylated Castor Oil, hydrogenated PEG-ylated Castor Oil, or a combination thereof.

According to embodiments, the alcohol of the hair tonic may be ethanol, or propanol, or a blend of ethanol and propanol. According to other embodiments, the alcohol of the hair tonic may be a blend of 70-50 parts-by-weight ethanol and/or propanol, and 30-50 parts-by-weight ethylene glycol and/or propylene glycol.

According to embodiments, the hair tonic further may comprise a supportive component. Examples of such supportive components include in particular vasodilatators, blood circulation stimulators, antioxidants, vitamins, provitamins. If present, the amount of the supportive component or combinations thereof will be in the range of from 0.0010 to 5.0, in particular from 0.010 to 3.0 percent-by-weight, based on the weight of the hair tonic.

The supportive component may comprise, for example, one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol (provitamin B5), omega-3 fatty acids, omega-6 fatty acids, folates, cannabidiol, saw palmetto, minoxidil, or combinations thereof.

According to embodiments, the supportive component includes a vasodilator selected from tocopherols (alpha-, beta-, gamma- or delta-tocopherol), in particular alpha-tocopherol, tocopherol esters (including, for example, tocopherol acetate, tocopheryl succinate, tocopherylnicotinate, tocopherylpoly(oxyethylene)-succinate), nicotinic acid, nicotinic amides, nicotinic esters, camphor; an antioxidant such as vitamin C or polyphenol(s); an inhibitor of testosterone-5-alpha-reductase such as the omega-6 fatty acid arachidonic acid; a substance providing a benefit to hair health such as nicotinic amide (niacinamide); or a combination thereof. According to embodiments, the supportive component includes tocopherol or tocopheryl acetate (vitamin E acetate), and/or niacinamide. Suitable vasodilatory plant extracts include burned or distilled nettle extract (Urtica Diocia), roast chestnut extract (Aesculus Hippocastanum) or Arnica extract (Arnica Montana).

According to specific embodiments, the hair tonic may include a supportive component selected from tocopherols, tocopherol esters, nicotininc acid, nicotinic amides, vasodilatory plant extracts, camphor, royal jelly, vitamin B3, vitamin B12, vitamin C, vitamin D2, vitamin D3, vitamin E, cannabidiol, minoxidil, or combinations thereof.

According to embodiments, the hair tonic further may comprise an oil component. The oil component may comprise, for example, peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof. If present, the oil component will be present in the hair tonic in an amount of from 0.010 to 0.50 , in particular from 0.025 to 0.25 percent-by-weight, based on the weight of the hair tonic.

According to embodiments, the oil component of the hair tonic further may be selected from peppermint oil, rosemary essential oil, thyme oil, cedarwood oil, bergamot oil, jojoba oil, hydrogenated ethylhexyl olivate, or a combination thereof. For example, the oil component of the hair tonic, if present, may include peppermint oil and/or hydrogenated ethylhexyl olivate.

According to embodiments, the hair tonic further may comprise a mineral component. If present, a preferred mineral component of the hair tonic is, for example, zinc in a concentration of 0.0050-0.50 molar.

According to embodiments, the hair tonic further may comprise one or more cosmetically acceptable additives selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents, or combinations thereof. The total amount of these cosmetically acceptable additives may within a range of from 0.010 to 5.0, conveniently within a range of from 0.010 to 3.0 percent-by-weight, based on the weight of the hair tonic.

Examples and individual amount ranges for these cosmetically acceptable additives are indicated above in Chapter 5. According to embodiments, the hair tonic may comprise a moisturizer and/or a cooling agent. According to embodiments, the moisturizer may be selected from niacinamide, sodium PCA, glycine. According to embodiments, the cooling agent may be selected from menthol, menthyl lactate, isopulegol.

According to embodiments, the hair tonic may be essentially free of quaternary ammonium compounds, silicones, or a combination thereof. According to particular embodiments, the hair tonic may be free of quaternary ammonium compounds, silicones, or a combination thereof.

According to embodiments, the hair tonic may be essentially free of biotin, unsaturated fatty acids, non-film-forming polymers, or a combination thereof. In particular embodiments, the hair tonic may be free of biotin, unsaturated fatty acids, non-film-forming polymers, or a combination thereof.

For example, the hair tonic may be essentially free or be free of quaternary ammonium compounds, silicones, biotin, unsaturated fatty acids, non-film-forming polymers, or a combination thereof.

According to specific embodiments, the hair tonic consists essentially of:
0.90 to 1.20 percent-by-weight Sandal Pentanol,
0.90 to 1.20 percent-by-weight caffeine,
0.10 to 0.25 percent-by-weight lauric acid (C12:0),
0.010 to 0.50 percent-by-weight fragrance,
0.10 to 0.50 percent-by-weight PEG-40 Hydrogenated Castor Oil,
0.010 to 0.10 percent-by-weight PEG-35 Castor Oil,
optionally, 0.050 to 3.5 percent-by-weight nicotinic acid, nicotinic amide, camphor, panthenol, or a combination thereof,
optionally, 0.00010 to 0.010 percent-by-weight of a cosmetically acceptable antioxidant,
optionally, up to 1.0, for example 0.010 to 1.0 percent-by-weight pH modifiers, preservatives and/or chelators,
optionally 0.010 to 1.0 percent-by-weight of a cooling agent,
40.0 to 65.0 percent-by-weight of ethanol, or propanol, or a blend of ethanol and propanol, or a blend of 70-50 parts-by-weight ethanol and/or propanol, and 30-50 parts-by-weight ethylene glycol and/or propylene glycol,
ad 100 percent-by-weight water.

According to specific embodiments, the hair tonic consists of:
0.90 to 1.20 percent-by-weight Sandal Pentanol,
0.90 to 1.20 percent-by-weight caffeine,
0.10 to 0.25 percent-by-weight lauric acid (C12:0),
0.010 to 0.50 percent-by-weight fragrance,
0.10 to 0.50 percent-by-weight PEG-40 Hydrogenated Castor Oil,
0.010 to 0.10 percent-by-weight PEG-35 Castor Oil,
optionally, 0.050 to 3.5 percent-by-weight nicotinic acid, nicotinic amide, camphor, panthenol, or a combination thereof,
optionally, 0.00010 to 0.010 percent-by-weight of a cosmetically acceptable antioxidant,
optionally, up to 1.0, for example 0.010 to 1.0 percent-by-weight pH modifiers, preservatives and/or chelators,
optionally 0.010 to 1.0 percent-by-weight of a cooling agent,
40.0 to 65.0 percent-by-weight of ethanol, or propanol, or a blend of ethanol and propanol, or a blend of 70.50 parts-by-weight ethanol and/or propanol, and 30-50 parts-by-weight ethylene glycol and/or propylene glycol,
ad 100 percent-by-weight water.

According to specific embodiments, the hair tonic consists essentially of:
0.950 to 1.050 percent-by-weight Sandal Pentanol,
0.950 to 1.050 percent-by-weight caffeine,
0.180 to 0.220 percent-by-weight lauric acid (C12:0),
2.30 to 2.70 percent-by-weight nicotinic acid or nicotininc amide,
0.10 to 0.40 percent-by-weight fragrance,
0.10 to 0.250 percent-by-weight PEG-40 Hydrogenated Castor Oil,
0.030 to 0.060 percent-by-weight PEG-35 Castor Oil,
0.00010 to 0.010 percent-by-weight antioxidant selected from tocopherol, tocotrienol(s), tocopheryl acetate,
optionally, up to 0.10, for example 0.010 to 0.10 percent-by-weight pH modifiers, preservatives and/or chelators,
50.0 to 55.0 percent-by-weight ethanol,
ad 100 percent-by-weight water.

According to specific embodiments, the hair tonic consists of:
0.950 to 1.050 percent-by-weight Sandal Pentanol,
0.950 to 1.050 percent-by-weight caffeine,
0.180 to 0.220 percent-by-weight lauric acid (C12:0),
2.30 to 2.70 percent-by-weight nicotinic acid or nicotininc amide,
0.10 to 0.40 percent-by-weight fragrance,
0.10 to 0.250 percent-by-weight PEG-40 Hydrogenated Castor Oil,
0.030 to 0.060 percent-by-weight PEG-35 Castor Oil,
0.00010 to 0.010 percent-by-weight antioxidant selected from tocopherol, tocotrienol(s), tocopheryl acetate,
optionally, up to 0.10, for example 0.010 to 0.10 percent-by-weight pH modifiers, preservatives and/or chelators,
50.0 to 55.0 percent-by-weight ethanol,
ad 100 percent-by-weight water.

### 9 Methods and Uses

The compositions or the hair tonics according to the present disclosure may be employed according to a method wherein the composition or hair tonic is applied to scalp. Conveniently, the composition or hair tonic may be applied to the scalp and subsequently or simultaneously be gently worked in.

The compositions or hair tonics according to the present disclosure are applied in an amount sufficient to obtain the desired effect. In particular, the compositions or hair tonics may be applied applied to the scalp in order to deliver an effective amount of the active ingredient combination. For example, the compositions or hair tonics may be applied applied to the scalp in order to deliver a total amount of at least 0.25 g of Sandal Pentanol, caffeine and C10-C18 fatty acid(s) per day. According to embodiments, the compositions or hair tonics may be applied applied to the scalp in order to deliver a total amount of at least 0.50 g of Sandal Pentanol, caffeine and C10-C18 fatty acid(s) per day. For example, the compositions or hair tonics may be applied applied to the scalp in two or three applications per day in order to deliver a total amount of at least 0.80 g of Sandal Pentanol, caffeine and C10-C18 fatty acid(s) per day.

The compositions or hair tonics according to the present disclosure are left on the scalp for sufficient time to obtain the desired effect. In particular, the compositions or hair tonics may be leave-on products, which after application to hair and/or scalp are not—or at least not immediately—rinsed or washed out.

Further subject matter of the present disclosure is the use of the compositions or hair tonics for promoting natural mammalian hair growth and/or reducing mammalian hair loss. According to embodiments, the compositions or hair tonics may be used for treating alopecia in mammals, in particular in humans.

### EXAMPLES

The following examples should illustrate the invention claimed below in greater detail, but the details of these examples should not be considered as further limiting the claims appended hereinbelow.

### Example 1

Example 1 demonstrates the hair loss reduction achieved by a composition according to the invention in a Hair Pull Test.

TABLE 1
Formulations

| Raw Material | Formulation 1 | | Formulation 2 | | Formulation 3 | |
|---|---|---|---|---|---|---|
| Sandal Pentanol | 1.0 | g | | | 1.0 | g |
| Lauric Acid | 0.2 | g | 0.2 | g | | |
| Caffeine | 1.0 | g | 1.0 | g | | |
| PEG-40 Hydrogenated | 0.18 | g | 0.18 | g | 1.53 | g |
| Castor Oil | | | | | | |
| PEG-35 Castor Oil | 0.0455 | g | 0.0455 | g | | |
| Ethanol | 53 | g | 53 | g | 18 | g |
| Perfume | 0.35 | g | 0.35 | g | | |
| Menthol | 0.25 | g | 0.25 | g | | |
| Disodium EDTA | | | | | 0.06 | g |
| Ethoxydiglycol | | | | | 0.5 | |
| Tocopheryl Acetate | | | | | 0.0002 | g |
| Citric Acid | | | | | 0.00005 | g |
| Water | to 100 | g | to 100 | g | to 100 | g |

### Hair Pull Test

This test is based on the concept of ‘gentle’ pulling of the hair to bring about shedding of telogen hair so it allows to roughly evaluate the intensity of the hair loss and to measure the resistance of the hair to traction. This method allows to measure the %-number of only loosely anchored hairs in the scalp and, the performance of the active ingredient combinations or compositions, respectively, on reducing hair loss. The ability of promoting hair growth, and/or inhibiting or delaying hair loss, respectively, will result in a higher number of hair which cannot be pulled out in this test.

The principle of the Hair Pull Test consists of slightly pulling about ˜60 hair on 3 delineated scalp areas and to score the hair loss.

On hair unwashed for 2 days, neither brushed nor combed within the 2 hours before examination, 3 areas of the scalp (fronto-temporal, parietal and occipital) are chosen.

A clump of about ˜60 hair per area is taken between the thumb and the forefinger and slightly pulled. The hairs lost with the pulling are collected and counted.

The Hair Pull Test is carried out by a technician at the investigating centre, at the following experimental times:
before any product application on D1/T0,
after 8 consecutive weeks of product use (D56),
after 16 consecutive weeks of product use (D112).

### Expression of the Results

All the test subjects included in the study are taken into account for Hair Pull Test as long as they were submitted to the last examination at the defined time.

For each product, the following data was calculated:
average and the standard deviation at D1/T0, D56 and D112,
variations in percentage D56 vs D1/T0 and D112 vs D1/T0,
distribution of difference (D56-D1/T0 and D112-D1/T0) by a Shapiro-Wilk test, at risk of 1%.

The significance is calculated according to p-value of bilateral Student “t” test in pairs, in case of normality of the distribution or the Wilcoxon test in the adverse case.

The variations are evaluated as statistically significant if p<0.05.

Table 2 summarizes the effectiveness results for hair treatment with:
Formulation 1 (invention: 1% Sandalore, 1% Caffeine, 0.2% Lauric Acid)
Formulation 2 (comparative:1% Caffeine, 0.2% Lauric Acid, no Sandalore)
Formulation 3 (comparative: 1% Sandalore only)

TABLE 2
Hair Pull Test Results

| | | Percentage Variation vs. | |
|---|---|---|---|
| | | D1 (T0) | Significance |
| Samples | Time | (equals hair loss reduction) | (p < 0.05) |
| Placebo | D1 (T0) | na | na |
| | D56 | −1.8 | Non-Significant |
| | D112 | −2.6 | Non-Significant |
| Formulation 1 | D1 (T0) | na | na |
| | D56 | −20.1 | Significant |
| | D112 | −27.7 | Significant |
| Formulation 2 | D1 (T0) | na | na |
| | D56 | −17.2 | Significant |
| | D112 | −21.6 | Significant |
| Formulation 3 | D1 (T0) | na | na |
| | D84 | −17.1 | Significant |

The data indicate an statistically significant hair loss reduction up to 20.1% and 27.7% after 56 days (8 weeks) or 112 days (16 weeks), respectively, for a composition according to the invention (Formulation 1). The composition without Sandalore (Formulation 2) and the composition containing just 1% Sandalore (Formulation 3) show a significantly lower effectiveness.
For Formulation 2, a hair loss reduction of 17.2% after 56 days (8 weeks) and 21.6% after 112 days (8 weeks) of usage, respectively, demonstrates lower effectiveness as compared to Formulation 1.
For Formulation 3, the hair loss reduction (−17.1%) is still lower even after 84 days (12 weeks) of application as compared to the composition according to the invention.

### Example 2

Example 2 demonstrates the hair growth achieved by a composition according to the invention as determined by prolongation of the anagen phase in ex vivo skin treatment.

Human full thickness scalp skin punches (6 mm diameter) with hair from the occipital region were obtained from a 34 year old female donor. The dissected human punches were cultured at 37° C. with 5% CO2 in a minimal media of William's E media (Gibco, Life Technologies) supplemented with 10 ng/ml hydrocortisone (Sigma Aldrich), 10 μg/ml insulin (Sigma Aldrich) and 1% penicllin/streptomycin (Gibco) to make William's Complete Media (WCM) modified as previously described (Edelkamp et al., Molecular Dermatology 2020, Langan et al., Exp Dermatol 2015). After 24 h resting culture (day 0), two topical treatments were Performed (day 1 and 3). The medium was changed at day 3. Punches were embedded in OCT Compound (Optimal Cutting Temperature Compound) after 5 days (day 5) of ex vivo culture and cryosectioned (7 μm sections). The experimental groups for the topical treatments are outlined in Table 3.

TABLE 3
Experimental Groups

| experimental group | # samples | treatment |
|---|---|---|
| #1 | n = 11 | 0.001% DMSO |
| #2 | n = 16 | 500 μM Sandalore |
| | | (corresponding to 1%) |
| #3 | n = 15 | 500 μM Sandalore + 1% Caffeine |
| #4 | n = 16 | 500 μM Sandalore + 1% Caffeine + |
| | | 0.2% Lauric Acid |
| #5 | n = 13 | 5% Minoxidil |

Hair cycle staging and hair cycle score are perfomed at the end of the culture and can be determined according to several established macroscopic and microscopic paremeters (Langan et al., Exp Dermatol 2015; Oh et al., JID 2016).

Hair follicles are microscopically evaluated for the hair cycle staging using Masson-Fontana histochemistry and Ki-67/TUNEL immunostaining as previously described (Kloepper at al., Exp Dermatol 2010; Langan et al., Exp Dermatol 2015). For each experimental group, the percentage of hair follicles in each hair cycle phase was calculated and is shown below.

TABLE 4
Microscopic hair cycle staging

| | | % of hair follicles in | |
|---|---|---|---|
| | | | early catagen |
| experimental group | # samples | anagen phase | phase |
| #1 (vehicle) | n = 11 | 36 | 64 |
| #2 (Sand) | n = 16 | 63 | 38 |
| #3 (Sand + Caff) | n = 15 | 60 | 40 |
| #4 (Sand + Caff + LA) | n = 16 | 75 | 25 |
| #5 Minox) | n = 13 | 77 | 23 |

TABLE 5
Hair cycle score (a lower number indicates more
hairs are in the anagen/growth phase)

| | | hair cycle score |
|---|---|---|
| experimental group | # samples | [arbitrary units] ± SEM |
| #1 (vehicle) | n = 11 | 163.6 ± 15.21 |
| #2 (Sand) | n = 16 | 137.5 ± 12.5 |
| #3 (Sand + Caff) | n = 15 | 140.0 ± 13.09 |
| #4 (Sand + Caff + LA) | n = 16 | 125.0 ± 11.18 |
| #5 Minox) | n = 13 | 123.1 ± 12.16 |

hair cycle score: 100 = anagen; 200 = early catagen; 300 = mid catagen; 400 = late catagen

The data in Tables 4 and 5 demonstrate that topical application of Sandalore on ex vivo cultured scalp skin prolongs anagen. A combination treatment with caffeine and lauric acid further enhances this effect.

1. Composition, comprising sandal pentanol, caffeine, one or more saturated and/or unsaturated C10-C18 fatty acids, and a solvent.

2. The composition of claim 1, wherein the C10-C18 fatty acid(s) is/are selected from capric acid (C10:0), lauric acid (C12:0), myristic acid (C14:0), isomyristic acid (C14:0), palmitic acid (C16:0), palmitoleic acid (C16:1), stearic acid (C18:0), oleic acid (C18:1), linoleic acid (C18:2), linolenic acid (C18:3), parinaric acid (C18:4), petroselinic acid (C18:1), or combinations thereof.

3. The composition of claim 1 or 2, wherein the C10-C18 fatty acid(s) comprise lauric acid (C12:0), myristic acid (C14:0), or a combination thereof.

4. The composition of any of claims 1 to 3, wherein the C10-C18 fatty acid(s) comprise palmitoleic acid (C16:1), oleic acid (C18:1), linoleic acid (C18:2), linolenic acid (C18:3), parinaric acid (C18:4), petroselinic acid (C18:1), or a combination thereof.

5. The composition of any of claims 1 to 3, wherein the composition is essentially free of unsaturated C10-C18 fatty acids.

6. The composition of any of the preceding claims, wherein the C10-C18 fatty acid(s) is/are present in the form of free acid, alkali metal salt, alkaline earth metal salt, ammonium salt or combination thereof, in particular wherein the fatty acid(s) is/are present in the form of free acid(s).

7. The composition of any of the preceding claims, wherein the sandal pentanol is present in an amount of from 0.010 to 10.0 percent-by-weight, in particular in an amount of 0.10 to 2.0 percent-by-weight, based on the weight of the composition.

8. The composition of any of the preceding claims, wherein the caffeine is present in an amount of from 0.0010 to 10.0 percent-by-weight, in particular in an amount of 0.10 to 2.0 percent-by-weight, based on the weight of the composition.

9. The composition of any of the preceding claims, wherein the one or more C10-C18 fatty acid(s) is/are present in an amount of from 0.010 to 10.0 percent-by-weight, in particular in an amount of 0.10 to 2.0 percent-by-weight, based on the weight of the composition.

10. The composition of any of the preceding claims, wherein the sandal pentanol is present in an amount of from 0.50 to 2.0 percent-by-weight, the caffeine is present in an amount of from 0.50 to 2.0 percent-by-weight, and the one or more C10-C18 fatty acid(s) is/are present in an amount of from 0.10 to 2.0 percent-by-weight, each based on the weight of the composition.

11. The composition of any of the preceding claims, wherein the sandal pentanol is present in an amount of from 0.80 to 1.50 percent-by-weight, the caffeine is present in an amount of from 0.80 to 1.50 percent-by-weight, and the one or more C10-C18 fatty acid(s) is/are present in an amount of from 0.10 to 1.0 percent-by-weight, each based on the weight of the composition.

12. The composition of any of the preceding claims, further comprising a supportive component, in particular a supportive component selected from vasodilatators, blood circulation stimulators, antioxidants, vitamins, provitamins, and combinations thereof.

13. The composition of claim 12, wherein the supportive component is present in an amount of from 0.0010 to 5.0, in particular from 0.010 to 3.0 percent-by-weight, based on the weight of the composition.

14. The composition of claim 12 or 13, wherein the supportive component comprises one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol, omega-3 fatty acids, omega-6 fatty acids, folates, cannabidiol, saw palmetto, minoxidil, or combinations thereof.

15. The composition of any of the preceding claims, further comprising an oil component selected from peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof.

16. The composition of claim 15, wherein the oil component is present in an amount of from 0.0010 to 1.0, in particular from 0.010 to 0.50 percent-by-weight, based on the weight of the composition.

17. The composition of any of the preceding claims, further comprising a mineral component selected from iron, zinc, or a combination thereof.

18. The composition of claim 17, wherein the mineral component is present in 0.0050-0.50 molar concentration.

19. The composition of any of the preceding claims, wherein the solvent is water-based, alcohol-based or water-alcohol-based.

20. The composition of any of the preceding claims, wherein the solvent comprises water and an alcohol selected from ethanol, propanol, glycerol, ethylene glycol, propylene glycol or a combination thereof.

21. The composition of any of the preceding claims, further comprising a solubilisation enhancer.

22. The composition of claim 21, wherein the solubilisation enhancer comprises one or more surfactants.

23. The composition of any of the preceding claims, having a pH within the range of from 3.0 to 8.0, in particular within the range of from 4.0 to 6.5, such as within the range of from 4.5 to 6.0.

24. The composition of any of the preceding claims, further comprising one or more cosmetically acceptable additives selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents, or combinations thereof.

25. The composition of any of the preceding claims, wherein the composition is essentially free of biotin.

26. The composition of any of the preceding claims, wherein the composition is essentially free of quaternary ammonium compounds.

27. The composition of any of the preceding claims, wherein the composition is essentially free of silicones.

28. The composition of any of the preceding claims, wherein the composition is essentially free of non-film-forming polymers.

29. The composition of claim 28, wherein the composition is essentially free of acrylates/Steareth-20 methacrylate copolymer, carbomer, polyacrylamide, hydroxyethylcellulose, Xanthan Gum.

30. The composition of any of the preceding claims, comprising 70.0 to 99.5 percent-by-weight, in particular 90.0 to 99.0 percent-by-weight, for example 92.0 to 98.0 percent-by-weight, based on the weight of the composition, water-alcohol-based solvent.

31. The composition of claim 30, comprising water in an amount of at least 10 percent-by-weight, based on the weight of the composition, and alcohol in an amount of at least 20 percent-by-weight, based on the weight of the composition.

32. The composition of claim 30 or 31, wherein the solvent comprises an alcohol selected from ethanol, n-propanol, isopropanol, glycerol, ethylene glycol, propylene glycol or a combination thereof.

33. The composition of any of claim 30 to 32, wherein the composition is a hair tonic.

34. The composition of any of claim 1 to 29, comprising 70.0 to 99.5 percent-by-weight, in particular 90.0 to 99.0 percent-by-weight, for example 92.0 to 98.0 percent-by-weight, based on the weight of the composition, of a blend comprising starch particles and an alcohol-based solvent.

35. The composition of claim 34, wherein the blend further comprises a propellant.

36. The composition of claim 34 or 35, wherein the blend further comprises inorganic particles.

37. The composition of any of claims 34 to 36, wherein the blend comprises, based on the weight of the blend, 5.0 to 12.0 percent-by-weight starch particles, 30.0 to 50.0 percent-by-weight alcohol-based solvent, 40.0 to 60.0 percent-by-weight propellant, and optionally 0.50 to 1.0 percent-by-weight inorganic particles.

38. The composition of any of claims 34 or 37, wherein the blend comprises, based on the weight of the blend, 7.0 to 10.0 percent-by-weight starch particles, 33.0 to 38.0 percent-by-weight alcohol-based solvent, 52.0 to 60.0 percent-by-weight propellant, and optionally 0.60 to 0.90 percent-by-weight inorganic particles.

39. The composition of any of claims 34 or 37, wherein the composition is a dry shampoo.

40. The composition of any of claims 1-29, wherein the composition is a ready-to-use scalp-and-hair treatment product selected from hair tonic, hair lotion, hair conditioner, hair cream, hair shampoo, dry shampoo, hair rinse, hair liquid, hair oil, hair pomade, hair foam, hair spray.

41. The composition of claim 40, wherein the product is a leave-on product.

42. A hair tonic, comprising:
0.90 to 1.20 percent-by-weight Sandal Pentanol,
0.90 to 1.20 percent-by-weight caffeine,
0.10 to 0.25 percent-by-weight of a C10-C18 fatty acid or an alkai metal salt thereof, wherein the fatty acid is selected from lauric acid (C12:0), myristic acid (C14:0), or a combination thereof,
0.010 to 0.60 percent-by-weight solubilisation enhancer,
0.010 to 0.50 percent-by-weight fragrance,
20.0 to 80.0 percent-by-weight alcohol,
ad 100 percent-by-weight water.

43. The hair tonic of claim 42, wherein the C10-C18 fatty acid or alkali metal salt thereof is lauric acid (C12:0) or sodium laurate.

44. The hair tonic of claim 42 or 43, wherein the solubilisation enhancer is selected from PEG-ylated Castor Oil, hydrogenated PEG-ylated Castor Oil, or a combination thereof.

45. The hair tonic of any of claims 42 to 44, wherein the alcohol is ethanol, or propanol, or a blend of ethanol and propanol.

46. The hair tonic of any of claims 42 to 44, wherein the alcohol is a blend of 70-50 parts-by-weight ethanol and/or propanol, and 30-50 parts-by-weight ethylene glycol and/or propylene glycol.

47. The hair tonic of any of claims 42 to 46, further comprising 0.050 to 3.50 percent-by-weight of a supportive component comprising one or more of tocopherols, tocotrienols, tocopherol esters, nicotinic acid, nicotinic amides, nicotinic esters, vasodilatory plant extracts, camphor, royal jelly, biotin, vitamin B3, vitamin B12, vitamin C, vitamin D2, vitamin D3, vitamin E, vitamin K1, vitamin K2, panthenol, omega-3 fatty acids, omega-6 fatty acids, folates, cannabidiol, saw palmetto, minoxidil, or combinations thereof.

48. The hair tonic of claim 47, wherein the supportive component is selected from tocopherols, tocopherol esters, nicotininc acid, nicotinic amides, vasodilatory plant extracts, camphor, royal jelly, vitamin B3, vitamin B12, vitamin C, vitamin D2, vitamin D3, vitamin E, cannabidiol, minoxidil, or combinations thereof.

49. The hair tonic of any of claims 42 to 48, further comprising 0.025 to 0.25 percent-by-weight of an an oil component selected from peppermint oil, rosemary essential oil, lavender essential oil, tea tree oil, ylang-ylang, thyme oil, cedarwood oil, clary sage essential oil, bergamot oil, jojoba oil, sunflower oil, hydrogenated ethylhexyl olivate, or a combination thereof.

50. The hair tonic of claim 49, wherein the oil component is selected from peppermint oil, rosemary essential oil, thyme oil, cedarwood oil, bergamot oil, jojoba oil, hydrogenated ethylhexyl olivate, or a combination thereof.

51. The hair tonic of any of claims 42 to 50, further comprising zinc in a concentration of 0.0050-0.50 molar.

52. The hair tonic of any of claims 42-51, further comprising 0.010 to 5.0 percent-by-weight of one or more cosmetically acceptable additives selected from perfumes, opacifiers, preservatives, pH modifiers, chelators, hair-care materials or skin-care materials, physiologically compatible silicone derivative compounds, light protecting agents, anti-flaking agents, hair luster-imparting agents, combability improving agents, defatting agents, moisturizers, conditioning agents, viscosity modifiers, cooling agents, or combinations thereof.

53. The hair tonic of claim 52, comprising a moisturizer and/or a cooling agent.

54. The hair tonic of any of claims 42-53, wherein the hair tonic is essentially free of quaternary ammonium compounds, silicones, or a combination thereof.

55. A hair tonic, consisting essentially of:
0.90 to 1.20 percent-by-weight Sandal Pentanol,
0.90 to 1.20 percent-by-weight caffeine,
0.10 to 0.25 percent-by-weight lauric acid (C12:0),
0.010 to 0.50 percent-by-weight fragrance,
0.10 to 0.50 percent-by-weight PEG-40 Hydrogenated Castor Oil,
0.010 to 0.10 percent-by-weight PEG-35 Castor Oil,
optionally, 0.050 to 3.5 percent-by-weight nicotinic acid, nicotinic amide, camphor, panthenol, or a combination thereof,
optionally, 0.00010 to 0.010 percent-by-weight of a cosmetically acceptable antioxidant,
optionally, up to 1.0, for example 0.010 to 1.0 percent-by-weight pH modifiers, preservatives and/or chelators,
optionally 0.010 to 1.0 percent-by-weight of a cooling agent,
40.0 to 65.0 percent-by-weight of ethanol, or propanol, or a blend of ethanol and propanol, or a blend of 70-50 parts-by-weight ethanol and/or propanol, and 30-50 parts-by-weight ethylene glycol and/or propylene glycol,
ad 100 percent-by-weight water.

56. A hair tonic, consisting essentially of:
0.950 to 1.050 percent-by-weight Sandal Pentanol,
0.950 to 1.050 percent-by-weight caffeine,
0.180 to 0.220 percent-by-weight lauric acid (C12:0),
2.30 to 2.70 percent-by-weight nicotinic acid or nicotininc amide,
0.10 to 0.40 percent-by-weight fragrance,
0.10 to 0.250 percent-by-weight PEG-40 Hydrogenated Castor Oil,
0.030 to 0.060 percent-by-weight PEG-35 Castor Oil,
0.00010 to 0.010 percent-by-weight antioxidant selected from tocopherol, tocotrienol(s), tocopheryl acetate,
optionally, up to 0.10, for example 0.010 to 0.10 percent-by-weight pH modifiers, preservatives and/or chelators,
50.0 to 55.0 percent-by-weight ethanol,
ad 100 percent-by-weight water.

57. A method for reducing or treating hair loss, wherein the method comprises applying the composition of any of claims 1 to 41 or the hair tonic of any of claims 42-55 to scalp.

58. The method of claim 57, wherein the composition of any of claims 1 to 41 or the hair tonic of any of claims 42-55 is applied to the scalp in an amount to deliver at least 0.25 g of Sandal Pentanol, caffeine and C10-C18 fatty acid(s) per day.

59. Use of the composition of any of claims 1 to 41 or of the hair tonic of any of claims 42-55 for promoting natural mammalian hair growth and/or reducing mammalian hair loss.

60. The composition of any of claims 1 to 41 or the hair tonic of any of claims 42-55 for use in the treatment of alopecia in mammals.
